(19) United States
(12) Patent Application Publication
(10) Pub. No.: US 2025/0272840 A1
(43) Pub. Date: Aug. 28, 2025

(54) A METHOD FOR ANALYZING BLOOD VESSELS

(71) Applicant: FERTIGO MEDICAL LTD., Zichron Ya'akov (IL)

(72) Inventor: Tsafrir KOLATT, Zichron Ya'akov (IL)

(73) Assignee: FERTIGO MEDICAL LTD., Zichron Ya'akov (IL)

(21) Appl. No.: 18/858,057

(22) PCT Filed: Apr. 25, 2023
(86) PCT No.: PCT/IL2023/050425

§ 371 Date: Oct. 18, 2024

Related U.S. Application Data
(60) Provisional application No. 63/334,932, filed on Apr. 26, 2022.

(51) Int. Cl. G06T 7/00; A61B 5/00; A61B 5/02

(52) U.S. Cl. CPC G06T 7/0016; A61B 5/02007; A61B 5/4306; G06T 2207/10024; G06T 2207/10048; G06T 2207/30101

(57) ABSTRACT

The present disclosure relates to methods of characterizing blood vessels at different depths within tissues.

### TECHNOLOGICAL FIELD

The present disclosure relates to methods for analyzing tissue and blood vessels.

### BACKGROUND ART

References considered to be relevant as background to the presently disclosed subject matter are listed below:
Acosta, A. A., Elberger, L., Borghi, M., Calamera, J. C., Chemes, H., Doncel, G. F., Kliman, H., Lema, B., Lustig,L., & Papier, S. (2000). Endometrial dating and determination of the window of implantation in healthy fertile women. Fertility and Sterility, 73(4), 788-798.
Bashkatov, A. N., Genina, E. A., & Tuchin, V. v. (2011). Optical properties of skin, subcutaneous, and muscle tissues: A review. Journal of Innovative Optical Health Sciences, 4(1), 9-38.
Chappell, J. C., Wiley, D. M., & Bautch, V. L. (2011). How blood vessel networks are made and measured. Cells Tissues Organs, 195(1-2), 94-107.
Chappell, J. C., Wiley, D. M., & Bautch, V. L. (2012). How Blood Vessel Networks Are Made and Measured. Cells Tissues Organs, 195(1-2), 94-107.
Du Cheyne, C., Smeets, M., & De Spiegelaere, W. (2021). Techniques used to assess intussusceptive angiogenesis: A systematic review. Developmental Dynamics, 250(12), 1704-1716.
Dubowy, R. L., Feinberg, R. F., Keefe, D. L., Doncel, G. F., Williams, S. C., McSweet, J. C., & Kliman, H. J. (2003). Improved endometrial assessment using cyclin E and p27. Fertility and Sterility, 80(1), 146-156.
Gambino, L. S., Wrefordm, N. G., Bertram, J. F., Dockery, P., Lederman, F., & Rogers, P. A. W. (2002). Angiogenesis occurs by vessel elongation in proliferative phase human endometrium. Human Reproduction, 17(5), 1199-1206.
Girling, J. E., Lederman, F. L., Walter, L. M., & Rogers, P. A. W. (2007). Progesterone, But Not Estrogen, Stimulates Vessel Maturation in the Mouse Endometrium. Endocrinology, 148(11), 5433-5441.
Jacques, S. L. (2013). Optical properties of biological tissues: a review. Physics in Medicine & Biology, 58(11), R37.
Logsdon, E. A., Finley, S. D., Popel, A. S., & MacGabhann, F. (2014). A systems biology view of blood vessel growth and remodelling. Journal of Cellular and Molecular Medicine, 18(8), 1491-1508.
Martinat-Botté, F., Renaud, G., Madec, F., Costiou, P., & Terqui, M. (2000). Ultrasonography and reproduction in swine (INRA). Intervet.
Murray, M. J., Meyer, W. R., Zaino, R. J., Lessey, B. A., Novotny, D. B., Ireland, K., Zeng, D., & Fritz, M. A. (2004). A critical analysis of the accuracy, reproducibility, and clinical utility of histologic endometrial dating in fertile women. Fertility and Sterility, 81(5), 1333-1343.
Noyes, R. W., Hertig, A. T., & Rock, J. (1950). Dating the Endometrial Biopsy. Fertility and Sterility, 1(1), 3-25.

Acknowledgement of the above references herein is not to be inferred as meaning that these are in any way relevant to the patentability of the presently disclosed subject matter.

### BACKGROUND

Blood Vessels are generated, grow and die regularly within organisms as part of their perpetual change: growth process, injury, occurrences of pathological conditions, apoptosis, necrosis and more. Hence, monitoring of blood vessel distribution could be of value in various tissue that are characterized by increased number of blood vessels, for example, skin, eye, intestine and uterine endometrium.

Measurement of different features of blood vessels, such as diameter, direction, tortuosity, spectrum, and average color, are feasible by imaging blood vessels at various wavelengths and techniques.

### GENERAL DESCRIPTION

The present disclosure provides in accordance with some aspects, a method for characterizing at least one feature of blood vessels at different depths within a tissue of a mammalian subject, the method comprising:
(i) identifying blood vessels in an image obtained from a surface of said tissue, and
(ii) determining for the identified blood vessels at least one feature of said identified blood vessels, a color index ( [symbol] ) or a combination thereof, wherein the color index differs in different depths within the tissue.

The present disclosure provides in accordance with some aspects, a method for characterizing at least one feature of blood vessels at different depths within a tissue of a mammalian subject, the method comprising:
(i) identifying blood vessels in an image obtained from a surface of said tissue,
(ii) determining for the identified blood vessels at least one feature of said identified blood vessels, a color index ( [symbol] ) or a combination thereof, and (iii) repeating step (i) and/or step (ii) in at least one temporarily separated time point, wherein the color index differs in different depths within the tissue.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic representation of the endometrium, and the tissues abut it, along with a basic morphology of blood vessels.

FIG. 2 is a schematic view of the uterus tissue layers and coordinates, with z=0 indicating the uterus surface, the endometrium tissue is considered to include the functional layer and the basal layer.

FIG. 3 is a theoretical graph showing penetration depth in endometrial tissue (where the signal drops by e⁻²) as function of wavelength of visible light.

FIG. 4 is a theoretical graph showing endometrium blood vessels color ratio as function of blood vessel depth for classical epithelial/mucousal tissue attenuation parameters.

FIGS. 5A to 5F are graphs showing theoretical sets of blood vessel diameter Probability Distribution Functions (PDFs) as function of depth for two competing evolution models (tracks) of blood vessels, FIGS. 5A-5C show blood vessels evolution led primarily by elongation, followed by capillary growth through splitting angiogenesis, FIGS. 5D-5F show a model by which blood vessels evolution of newly found layers is primarily governed by sprouting of newly formed capillaries, FIGS. 5A and 5D represent shallow layers (i.e., close to the tissue surface) and FIGS. 5B, 5C, 5E and 5F represent deeper layers (tissue depth).

FIGS. 6A-6I are graphs showing two different evolution models for individual recorded blood vessel diameter PDFs as function of tissue depth, FIGS. 6A-6C show an exemplary schematic representation of blood vessels diameter PDFs as function of tissue depth at day “n” of the standardized cycle day, FIGS. 6D-6F show an exemplary theoretical blood vessels diameter PDFs as function of tissue depth at day “n+m” of the standardized cycle day for the above-mentioned recorded individual (on day n) and FIGS. 6F-6I show a competing, exemplary theoretical blood vessels diameter PDFs as function of tissue depth at day “n+m” of the standardized cycle day for a different evolution track.

FIGS. 7A and 7B are images of fresh ex-vivo endometial tissue (from swine), overlaid with the computerized identification of blood vessels, FIG. 7A shows all identified vessels, FIG. 7B shows identified vessels on the upper left corner only to allow assessment of the identification accuracy, each image long side equals about 4.9 mm of tissue slab.

FIGS. 8A to 8F are histograms showing normalized Probability Distribution Functions (PDFs) of blood vessel diameters within depth measure ((B+G)/R—an exemplary color index) in human endometrial tissues, calculated from 48 samples extracted from adult women, FIG. 8A shows PDF for the deepest endometrial layer, FIGS. 8B-8E show PDF for intermediate endometrial layers, FIG. 8F shows PDF for the most superficial layer, the relative number density of the vessels (N_{bv}) and the log-normal function model parameters are shown, data processed from 344 images of 2× magnification, each covers a few mm².

FIGS. 9A to 9F are histograms showing normalized Probability Distribution Functions (PDF) of blood vessel diameters within depth measure ((B+G)/R—an exemplary color index) in human endometrial tissues, calculated from 48 samples extracted from adult women, FIG. 9A shows PDF for the deepest endometrial layer, FIGS. 9B-9E show PDF for intermediate endometrial layers, FIG. 9F shows PDF for the most superficial layer, the relative number density of the vessels (N_{bv}) and the log-normal function model parameters are shown, data processed from 625 images of 4×, each covers a few mm².

FIG. 10 is a graph showing change of the lognormal fitting parameter @ in units of log(μm) for blood vessel diameter PDF as function of tissue depth within depth (color ratio) bins, the X axis shows the average (B+G)/R values representing the endometrial layer depth, whereas smaller values (on the x-axis) represent layers more distant from the lumen of the uterine cavity (“deeper”), distributions calculated from human 2× (X symbols) and 4× (square symbols) image samples, vertical errorbars are of the order of the symbol size.

FIGS. 11A and 11B are graphs showing log-normal fitting parameter ratios of the blood vessel diameter PDFs (w in log space, FIG. 11A) and ratio of the “width” (σ, in log, FIG. 11B) as a function of the cycle day, two symmetrial (in color ratio) populations of deep and superficial blood vessels PDFs were fitted for each cycle day cluster of patients such that for each cycle day the ratio of the result and log-normal variance and standard deviation were calculated, long lines are regression lines for the entire cycle duration; short lines are regression lines for the proliferative and secretory phases separately, cycle days are inferred by histology, the bins central values are (B+G)/R=0.5 and 1.5 (deep and supferficial, respectively).

FIGS. 12A to 12E are histograms showing normalized Probability Distribution Functions (PDF) of blood vessel diameters within depth measure ((B+G)/R—an exemplary color index) in four adult sows proven fertile, FIG. 12A shows the PDF for the deepest endometrial layer, FIGS. 12B-8D show PDF for intermediate endometrial layers FIG. 12E shows PDF for the most superficial credible layer, and also involves artifacts of misinterpreted fresh blood on the surface, the relative number density of the vessels can be read from the total number (Nbv, on the left) and the log-normal function model parameters are written on the right of each panel, data processed from 73 images altogether, each of a few mm² at 2× magnification.

FIG. 13 is a graph showing @ fitting parameter in units of log(μm) of the log-normal distribution model for the blood vessel diameter of swine a as function of the tissue depth (deep is smaller x-axis value), Y-errors are of the order of the symbol size.

### DETAILED DESCRIPTION OF EMBODIMENTS

Blood vessels play an important role in various physiological and pathological conditions, including for example, tissue development, tissue formation, and wound healing. Therefore, monitoring a three-dimensional (3D) blood vessel network in tissues as well as the changes over time in the 3D network, may provide valuable information regarding tissue condition, for example, growth, or recovery phase as well as insights as to the angiogenesis functionality of the blood vessels.

The present disclosure is based on the findings that differences in colors of blood vessels, as observed in images obtained from a tissue surface, can be used to determine the depth coordinates of the tissue and specifically the depth of blood vessels in the tissue.

Specifically, it was found that extracting color band information from images, including, inter alia, color values of red, blue, and green channels, for blood vessels may be used to reveal three-dimensional spatial distribution of blood vessels. As shown herein, the z-coordinate of the blood vessels was used to examine the relationship between the depth of the blood vessels and it's characteristics, including, inter alia, blood vessel width or diameter.

To that end and as described herein, in order to obtain physiological information of the tissue and the blood vessels within the tissue, the entire blood vessel population was treated as a statistical ensemble that was characterized by its probability distribution functions (e.g., size, tortuosity, length between bifurcations, spatial correlation, directional correlation, etc.). As shown herein, by treating the blood vessel population as a statistical ensemble, it was possible to differentiate between different depths of blood vessels, using their color attribute as obtained in visual light images. This approach differentiates between evolution paths when blood vessel diameter distribution is taken into account. The use of visible-light, non-destructive imaging also allowed monitoring of the blood vessel population evolution over a period of time within the same tissue.

Based on these findings, it was suggested that it is possible to characterize various features of the tissue, including, inter alia, features related to blood vessels, at different depth of a tissue and hence obtaining information on three-dimensional blood vessel network. In addition, it was suggested that it is possible to monitor changes with time in one or more features of the three-dimensional blood vessel network. This may be beneficial in the characterization of physiological process, conditions and disease that are associated with changes in blood vessels, including, inter alia, growth of blood vessels and destruction thereof.

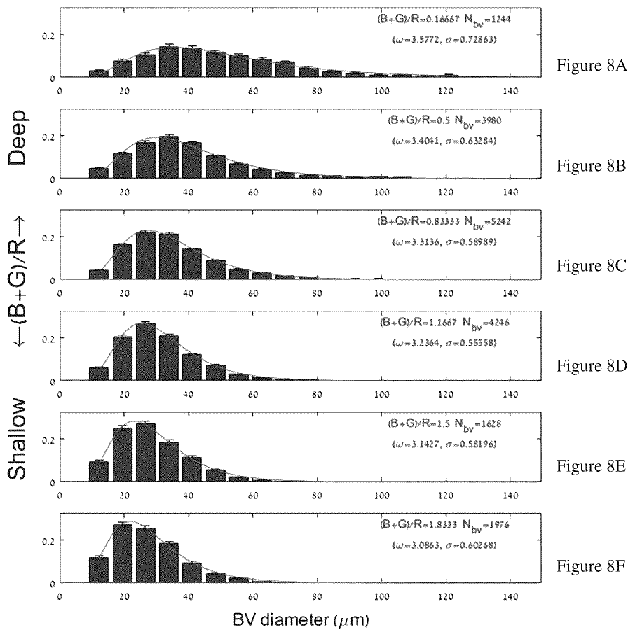

As shown for example in FIGS. 8A-8F, FIGS. 9A-9F and FIGS. 12A-12E, by employing the methods described herein, it was possible to monitor/characterize changes in blood vessel diameter as function of tissue layer's depth.

Hence, in accordance with its broadest aspect, the present disclosure provides a method for distinguishing different depths within a tissue. The method comprising analyzing color band distribution of blood vessels in one or more images obtained from the surface of the tissue to thereby distinguish between different depths of the tissue.

In accordance with some aspect, the present disclosure provides a method for characterizing the three-dimensional network of blood vessels, the method comprising analyzing blood vessels for the color values distribution in one or more images obtained from the surface of the tissue to thereby characterize the three-dimensional network of blood vessels.

In some examples, the method comprises obtaining one or more color images. In some examples, the method comprises obtaining one or more Infrared (IR) images.

In accordance with some other aspects, it is provided a method for characterizing three-dimensional network of blood vessels, the method comprising analyzing in one or more images obtained from the surface of the tissue, the color values distribution of red, blue and green channels of blood vessels identified in the images, to thereby characterize the three-dimensional network of blood vessels.

Characterizing the three-dimensional network of blood vessels in a tissue as used herein refers to characterizing one or more features of blood vessels, that may in turn provide information on physiological and/or pathological conditions of the tissue.

Hence, in accordance with some aspects, the present disclosure provides a method for characterizing at least one feature of blood vessels at different depths within a tissue of a mammalian subject.

The method comprises a step of identifying blood vessels in an image obtained from the surface of the tissue.

The method further comprises a step of analyzing the identified blood vessels. Analyzing the identified blood vessels comprises (i) determining at least one feature of the identified blood vessels, (ii) determining a color index for the identified blood vessels or (iii) a combination thereof.

The color index is denoted herein as [symbol] . As shown herein, it was found that the color index differs in different depths within the tissue.

In some embodiments, the color index is different at the tissue surface and at a different depth within the tissue, i.e. a deeper layer of the tissue. Hence, it was suggested that the color index may be used to indicate the tissue depth and accordingly to provide information on the one or more of the blood vessels features in different tissue depth.

Hence, in accordance with some aspects, the present disclosure provides a method comprising: (i) identifying blood vessels in one or more images, and (ii) determining one or more of at least one feature of the identified blood vessels, a color index ( [symbol] ) for the identified blood vessels or a combination thereof.

Hence, in accordance with some aspects, the present disclosure provides a method comprising: (i) identifying blood vessels in one or more images, and (ii) determining at least one feature of the identified blood vessels and a color index ( [symbol] ) for the identified blood vessels.

As described herein, the method allows to examine the relationship between the z-coordinate of a blood vessel as determined by the color index ( [symbol] ) and the blood vessels characteristics/features. In some embodiments, the method comprises obtaining one or more images of the tissue.

As noted herein, there are different imaging techniques that allow monitoring the blood vessels, including, for example a camera or an optic fiber.

The present disclosure is not limited to a specific imaging method and can be applicable to any spectroscopy method, provided that it allows a spatial two-dimensional image resolution that allows to resolve the vascular blood vessels, namely ˜10-100 μm.

As appreciated, the incident light spectrum is given by the selected illumination means. It may also be in conjuncture or replaced by the excited/spontaneous light emitting processes contributed by the blood vessels and the contained blood inside these vessels. Artificial light-emitting elements may also serve as illumination means (e.g., fluorophores, Q-dots, etc.). The wavelength dependent absorption and scattering mechanisms applied to the incident/emitted light are determined by the (partial) tissue slab it crosses until it hits the blood vessel (if not self-emitted) and on its way back to the collecting device (e.g., camera, fiber, fiber bundle). In case of an illumination source residing inside or nearby the blood vessel itself, only half the light path travel is taken into account.

In case of reflection, the reflection (magnitude, spectrum, polarization) off the target blood vessel is dictated by the blood vessel and the contained blood reflectance characteristics and the detection sensitivity is determined by the filters (including the camera, e.g., the RGB filters of the Bayer pattern), the camera optics and response function of its sensor.

As used herein, obtaining one or more of the images from the surface of the tissue refers to images that are obtained without physically penetrating the tissue. In other words, the method comprises obtaining the one or more of the images from the surface of the tissue reflects capturing a visual representation of the tissue without penetration or insertion or incision into the body such that the images are obtained from outside the tissue. In some other examples, the method comprises obtaining one or more images from the same distance to surface of the tissue.

Hence, the method provides in some examples, a non-invasive method that allows capturing (obtaining) images of a tissue surface without causing damage or injury to the tissue.

In some examples, one or more images are obtained by visible light or infra-red or any combination thereof.

In some other examples, one or more images are obtained by visible light.

In some further examples, one or more images are color images obtained by visible light spectroscopy.

In some embodiments, the method comprises obtaining one or more images in digital format.

Hence, in accordance with some aspects, the present disclosure provides a method for characterizing at least one feature of blood vessels at different depths within a tissue of a mammalian subject, the method comprising: (i) obtaining one or more images of the tissue from a surface of the tissue, (ii) identifying blood vessels in the one or more images, and (iii) determining one or more of (a) at least one feature of the identified blood vessels (b) a color index ( [symbol] ) for the identified blood vessels or (c) a combination thereof.

When referring to images obtained from the tissue surface, it should be understood that the tissue surface is considered to form a planar surface. The three spatial dimensions of the tissue are considered such that the z-coordinate represent the vessel depth or its average depth, with regard to the tissue outer surface and the planar x-y dimensions is represented by x- and y-coordinates.

The term “depth of the tissue” is used herein to refer to distance between layers of a tissue in the body and in the context of the present disclosure is defined as a distance between the surface of the tissue and a particular layer beneath it.

The blood vessel distance defined as the distance from the tissue surface may be determined statistically by the differential change of the spectrum it reflects or emits due to absorption and scattering processes the light undergoes during its passage through the tissue layers.

The depth of the tissue may depend (be affected) on various parameters including, inter alia, the tissue characteristics (for example degree of tissue transparency or complexity) and the dynamic range of the image capturing device (for example a camera).

In some examples, the depth of the tissue is at most about 10 cm, at times at most about 9 cm, at times at most about 8 cm, at times at most about 7 cm, at times at most about 6 cm, at times at most about 5 cm, at times at most about 4 cm, at times at most about 3 cm, at times at most about 2 cm, at times at most about 1 cm from the tissue surface.

In some examples, the depth of the tissue is at most about 1 cm, at times at most about 0.9 cm, at times at most about 0.8 cm, at times at most about 0.7 cm, at times at most about 0.6 cm, at times at most about 0.5 cm, at times at most about 0.4 cm, at times at most about 0.3 cm, at times at most about 0.2 cm, at times at most about 0.15 cm from the tissue surface.

In some examples, the depth of the tissue is between about 0.001 cm to about 10 cm, at times between about 0.001 cm to about 5 cm, at times between about 0.001 cm to about 3 cm, at times between about 0.001 cm to about 1 cm, at times between about 0.001 cm to about 0.7 cm, at times between about 0.001 cm to about 0.5 cm, at times between about 0.001 cm to about 0.1 cm.

In some examples, the depth of the tissue is between about 0.005 cm to about 10 cm, at times between about 0.007 cm to about 10 cm, at times between about 0.01 cm to about 10 cm, at times between about 0.03 cm to about 10 cm, at times between about 0.07 cm to about 10 cm, at times between about 0.1 cm to about 10 cm, at times between about 0.3 cm to about 10 cm, at times between about 0.7 cm to about 10 cm, at times between about 1 cm to about 10 cm.

In some examples, the depth of the tissue is about 0.005 cm, at times about 0.01 cm, at times about 0.03 cm, at times about 0.05 cm, at times about 0.07 cm, at times about 0.1 cm, at times about 0.3 cm, at times about 0.5 cm, at times about 0.7 cm, at times about 1 cm, at times about 3 cm, at times about 5 cm, at times about 7 cm, at times about 10 cm.

As described herein, the method comprises identifying one or more blood vessels. As used herein the term blood vessels encompasses an artery, a vein, a capillary or any combination thereof. The term one or more blood vessels as used herein may be denoted as time as blood vessel population.

The typical diameter range of blood vessels is shown in the Table below:

| Type | Diameter | |
|---|---|---|
| Arteries | ~0.1-10 | mm |
| Veins | ~1-10 | mm |
| Arterioles | ~20-30 | μm |
| Venules | ~8-100 | μm |
| Capillaries | ~5-10 | μm |

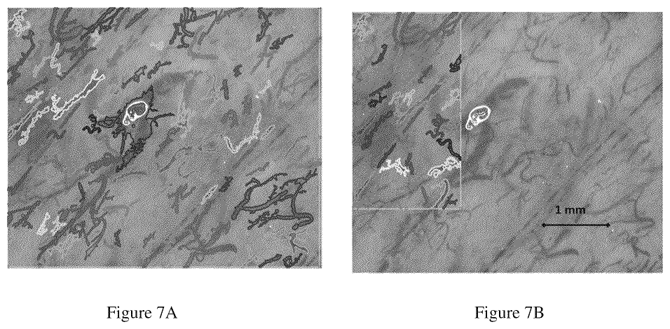

Blood vessels can be identified by employing different methods. For example, the blood vessels can be identified by the method described in the examples below and as shown in FIGS. 7A and 7B.

In some examples, the method comprises segmenting one or more blood vessels from an image to identify said one or more blood vessels. In other words, identifying blood vessels comprises segmenting the blood vessels in an image. Segmentation can be done by any method known in the art, including, for example, commercial software, such as MATLAB.

The identified one or more blood vessels may be subject to analysis as described herein.

In some embodiments, the method comprises analyzing the identified blood vessels in order to determine at least one feature of the blood vessels. As described herein and in accordance with some examples, determining at least one feature comprises calculating a probability distribution function (PDF) for at least one feature. In some examples, determining the at least one feature comprises obtaining a statistical ensemble for the at least one feature in a population of blood vessels.

In some embodiments, the present disclosure provides a method for characterizing at least one feature of a tissue of a mammalian subject at various depths of a tissue, wherein the tissue depth is as described herein, at most about 10 cm beneath the tissue surface. Hence, the method allows characterization of at least one feature of the tissue in different depths of the tissue ranging between the tissue surface and as described herein, at most about 10 cm beneath the tissue surface.

The selected feature may be any feature that is representative of a blood vessel, for example, a feature that differs in different depths of a tissue, i.e., a blood vessel feature that is different at a tissue surface and in a shallow layer.

In some embodiments, the blood vessel feature is or comprises of blood vessel structure. As appreciated, blood vessels are considered to have tube-like structures, typically made up of three layers. In some embodiments, the method comprises determining at least one feature of blood vessel structure.

In some embodiments, the blood vessel feature is or comprises blood vessel size. As appreciated, blood vessels vary in diameter, ranging from large arteries and veins that may be several centimeters in diameter, to tiny capillaries that are only a few micrometers in diameter. In some embodiments, the method comprises determining at least one feature of blood vessel size.

In some embodiments, the blood vessel feature is or comprises blood vessel elasticity. As appreciated, the three types of blood vessels may be characterized by different elasticity, for example, arteries have a higher degree of elasticity than veins, hence allowing them to expand and contract in response to changes in blood pressure. In some embodiments, the method comprises determining at least one feature of blood vessel elasticity.

In some embodiments, the blood vessel feature is or comprises blood flow in the blood vessels. As appropriated, blood vessels are responsible for the distribution of blood throughout the body such that the flow of blood is regulated by the contraction and relaxation of the smooth muscles in the walls of the blood vessels. In some embodiments, the method comprises determining at least one feature of blood flow.

In some embodiments, the blood vessel feature is or comprises vasoconstriction and vasodilation. As appreciated, the diameter of blood vessels can be controlled by the nervous system and various hormones through the processes of vasoconstriction and vasodilation. Vasoconstriction is the narrowing of blood vessels, while vasodilation is the widening of blood vessels. In some embodiments, the method comprises determining at least one feature of vasoconstriction and vasodilation.

In some embodiments, the at least one feature comprises at least one of blood vessel diameter, blood vessel width, blood vessel length, blood vessel degree of tortuosity, oxygen saturation level, flow pattern or any combination thereof.

In some embodiments, at least one feature comprises blood vessel width (w).

As described herein, it may be assumed that the blood vessel has a circular shape and hence the blood vessel width is as the blood vessel diameter.

As used herein, blood vessel width refers to the blood vessel diameter, which is the measurement of the distance across the blood vessel in one or more points of the blood vessel, for example, at its widest point.

In some examples, the blood vessel width (w) is calculated as:

$$\overline{w} = \frac{1}{l} {\int }_{0}^{l} w ( {l}^{′} ) d {l}^{′} .$$

The diameter of blood vessels may play an important role in determining the rate of blood flow through the vessel. Hence, the diameter may provide information on the blood flow.

As described herein, the depth of a tissue correlated with color information obtained from the blood vessels and specifically with a color index.

In some embodiments, the color index is calculated from the blood vessels color bands.

In some embodiments, the method comprises extracting color information from one or more images.

In some embodiments, the color index is calculated from a combination of the blood vessels red color, green color and red color.

In some other embodiments, the method comprises extraction color information from one or more images using red-green-blue (RGB) color model.

As appreciated, in a RGB color model, each color is represented as a combination of three primary colors: red, green, and blue values, usually ranging, for example, from 0 to 255 such that for example, red is represented as (255, 0, 0), green is (0, 255, 0), and blue is (0, 0, 255).

In some embodiments, the method comprises determining the distribution of red color value, green color value and blue color value in one or more blood vessels.

In some embodiments, the method comprises applying a pixel-by-pixel analysis on the identified blood vessels.

As appreciated, a pixel-by-pixel analysis refers to examination/interpretation of an image at an individual pixel level. In other words, a pixel-by-pixel analysis involves analyzing each pixel in an image and considering one or more of its color, intensity and texture.

In some embodiments, the method comprises applying a pixel-by-pixel analysis of the red color value, the green color value and the blue color value values in each one of the one or more blood vessels.

In some embodiments, the method comprises extracting a red color component (Rₚ) for each pixel in the identified blood vessels to obtain a set of red color values.

In some other embodiments, the method comprises extracting a green color component (Gₚ) for each pixel in the identified blood vessels to obtain a set of green color values.

In some further embodiments, the method comprises extracting a blue color component (Bₚ) for each pixel in the identified blood vessels to obtain a set of blue color values.

In some embodiments, the method comprises generating an output indicating the red color component values and their corresponding pixel locations within the one or more images.

In some embodiments, the method comprises generating an output indicating the blue color component values and their corresponding pixel locations within one or more images.

In some embodiments, the method comprises generating an output indicating the green color component values and their corresponding pixel locations within one or more images.

In some embodiments, the method comprises determining an average value for each one of red, green and blue values in each one of the one or more blood vessels.

In some embodiments, the method comprises calculating a mean value of the set of red color values to determine an average red color (R_{avg}) of the identified blood vessels. In some other embodiments, the method comprises calculating a mean value of the set of green color values to determine an average green color (G_{avg}) of the identified blood vessels. In some further embodiments, the method comprises calculating a mean value of the set of blue color values to determine an average blue color (B_{avg}) of the identified blood vessels.

As described herein, the color index (denoted herein as R) is a numerical value calculated for each blood vessel (identified blood vessel) and is considered herein to be indicative of the tissue depth.

In accordance with some embodiments, the color index is calculated from one or more combinations of R_{avg}, G_{avg} and B_{avg}.

In some examples, the color index is a dimensionless quantity and hence the combination of R_{avg}, G_{avg} and B_{avg} is given as any ratio of R_{avg}, G_{avg} and B_{avg}.

In accordance with some embodiments, the color index is calculated as (B_{avg}+G_{avg})/R_{avg} (Formula I).

In accordance with some other embodiments, the color index is calculated as B_{avg}/R_{avg} (Formula II).

In accordance with some other embodiments, the color index is calculated as G_{avg}/R_{avg} (Formula III).

As described herein, the color index has a different value in different depths of the tissue. FIG. 4 shows a model-based graph showing the changes in a color index calculated by Formula (I) as a function of the blood vessel depth suggesting that the color index is a sensitive measure that is associated (correlated) with the depth of the blood vessel. In other words, it was suggested that the color index has a different value at the tissue surface and at a deeper tissue layer.

By determining the tissue depth, i.e., the z coordinate, the present method may provide spatial coordinates of blood vessels in the tissue.

In some examples, the method is for determining spatial coordinates of the blood vessels within the tissue.

The spatial coordinates of blood vessels as used herein refer to the location of the blood vessel(s) in the body and specifically in the examined tissue, optionally, relative to other structures. As appreciated, spatial coordinates of blood vessels may be defined using a Cartesian coordinate system, which consists of three axes: x, y, and z, such that as described herein, the z-axis represents the depth or distance from a surface of the issue.

The information obtained by the methods described herein may be useful for determining various correlations, including one or more of spatial correlation, directional correlation, morphological correlation, functional correlation or any combination thereof. Typically, such correlations may be determined using statistical methods.

In some examples, the method is for determining spatial correlation of at least one feature of the blood vessels and/or of the spatial coordinates of the blood vessels.

As used herein, the term spatial correlation refers to the excess (over random) probability of finding at least one feature of the blood vessels and/or spatial coordinates of the blood vessels given the location and at least one feature of another blood vessel.

In other words, it characterizes the pattern of the blood vessels' spatial distribution within the tissue or the same for at least one feature thereof.

In some examples, the method is for determining directional correlation of at least one feature of the blood vessels and/or of the spatial coordinates of the blood vessels.

As used herein, the term directional correlation refers to the excess (over random) probability of finding at least one feature of the blood vessels and/or spatial orientation (“direction”) of the blood vessels given the location and at least one feature of another blood vessel in a given orientation (“direction”).

In some examples, the method is for determining morphological correlation of at least one feature of the blood vessels.

As used herein, the term morphological correlation refers to the degree to which the shape or structure of the blood vessels as determined by at least one feature of the blood vessels is correlated with other blood vessels (or other features).

In some examples, the method is for determining functional correlation of at least one feature of the blood vessels.

As used herein, the term functional correlation refers to the degree to which the function of the blood vessels as determined by at least one feature of the blood vessels is correlated with other blood vessels (or other features).

As noted herein, the ability to distinguish between different depths of a tissue allows gathering information on one or more features of the blood vessels in different depths of the tissue in order to provide information on the tissue physiology.

Hence, in accordance with some embodiments, the method is for characterizing physiological process in the tissue. The physiological process may be any process associated with blood vessels as well as any process associated with changes in blood vessels characteristics.

In some embodiments, the physiological process is one or more of blood pressure regulation, blood clotting, differentiation, neovascularization, angiogenesis and apoptosis or any related processes.

In some embodiments, the physiological process is blood pressure regulation.

In some embodiments, the method is for characterizing blood pressure or any related process in the tissue.

Blood pressure regulation as used herein refers to a physiological process that controls the force or pressure of blood against the walls of arteries as it circulates through the body. Blood pressure is regulated by various processes, such as adjusting the diameter of blood vessels. Related processes associated with blood pressure include, for example, vasoconstriction. Vasoconstriction is a physiological process associated with decreased width or narrowing of blood vessels.

In some embodiments, the physiological process is blood clotting.

In some embodiments, the method is for characterizing blood clotting or any related process in the tissue.

Blood clotting, also known as coagulation, as used herein refers to a process by which the body stops bleeding by forming a plug of fibrin and platelets at the site of injury.

In some embodiments, the physiological process is differentiation.

In some embodiments, the method is for characterizing differentiation or any related process in the tissue.

In some embodiments, the physiological process is neovascularization.

In some embodiments, the method is for characterizing neovascularization or any related process in the tissue.

In some embodiments, the physiological process is angiogenesis.

In some embodiments, the method is for characterizing angiogenesis or any related process in the tissue.

Angiogenesis as used herein refers to a process by which new blood vessels form from existing blood vessels. Generation of new blood vessels may be via sprouting, intussusception (splitting angiogenesis) or elongation.

Neovascularization as used herein refers to a pathological type of angiogenesis, such as in diseases like cancer, diabetic retinopathy, and age-related macular degeneration, where the growth of new blood vessels can contribute to tissue damage and disease progression.

In some embodiments, the method is for characterizing tissue growth and repair.

In some embodiments, the method is for characterizing sprouting and branching of new blood vessels from preexisting blood vessels.

In some embodiments, the physiological process is apoptosis.

In some embodiments, the method is for characterizing apoptosis in the tissue.

In some embodiments, the method is for characterizing wound healing.

In some embodiments, the method is for characterizing tumor progression.

Characterization of physiological processes associated with blood vessels and specifically changes in blood vessels features may be relevant for diagnosis of various conditions/disease.

In some embodiments that may be considered as aspects of the present disclosure, the method is a method for diagnosis a condition or a disease associated with blood vessels.

In some embodiments, the method is for diagnosis a condition associated with tissue growth and repair.

In some embodiments, the method is for diagnosis a condition associated with sprouting and branching of new blood vessels from preexisting blood vessels.

In some embodiments, the method is for diagnosis a condition associated with apoptosis.

In some embodiments, the method is for diagnosis a condition associated with apoptosis in the tissue.

In some embodiments, the method is for diagnosis a condition associated with wound healing.

In some embodiments, the method is for diagnosis a condition associated with tumor progression. In some embodiments, the method is for diagnosis proliferative disorder.

As appreciated, analysis of the 3D blood vessels network and consequently obtaining information on a physiological process and optionally regarding a condition or disease associated with this process over time (i.e., in at least two separated time points) is also important as it allows comparison of the 3D blood vessels network and/or at least one feature as described herein at different times and hence measure their evolution.

Hence, the present disclosure provides a method for characterizing blood vessel evolution over time.

As used herein, blood vessel evolution refers to the process by which blood vessels are developed and changed over time through normal as well as abnormal mechanisms. As appreciated, while in some cases, evolution of blood vessels contributes to development of a highly efficient circulatory system, capable of delivering oxygen and nutrients to all parts of the body, in some cases such evolution may be associated with improper development and even diseases and other health problems.

In some embodiments, the physiological process is associated with changes in blood vessels and the blood vessels' spatial distribution over time.

In some embodiments, the method is for characterizing changes in blood vessels with time.

Hence, the present disclosure provides dynamic measurements of the least one feature in the tissue.

As used herein the term dynamic measurements refer to the ability to measure changes in the tissue and specifically to at least one blood vessel feature, over time. As further described herein, such measurements may be used to monitor tissue function and to diagnose and/or treat disease.

In some embodiments, the method comprises determining the at least one feature in at least two temporarily separated time points to monitor changes in blood vessels in the different depths within the tissue over time.

As shown in FIGS. 11A and 11B, changes were observed in the calculated ratio at different days of the cycle. Specifically, the slope change of the ratio evolution shows a pivotal point at or near day 14 (“ovulation”), which separated the two cycle phases.

Hence, in accordance with some aspects, the present disclosure provides a method for characterizing at least one feature of blood vessels at different depths within a tissue of a mammalian subject, the method comprising: (i) identifying blood vessels in an image obtained from a surface of said tissue, (ii) determining one or more of at least one feature of the identified blood vessels, a color index for the identified blood vessels or any combination thereof, wherein the color index differs in different depths within said tissue and (iii) repeating steps (i) and/or (ii) in at least one more temporally separated time point.

The two temporarily separated time points as used herein encompass two distinct moments in time that are separated by a period of time.

The images obtained at the two temporarily separated time points may provide a continuous monitoring of the tissue over time or an intermittent monitoring of the tissue over time.

In some examples, the method provides a continuous monitoring of the tissue. In some examples, continuous monitoring allows characterization of blood flow.

In some examples, the method provides an intermittent monitoring of the tissue. Intermittent monitoring, i.e. a non-continuous monitoring of the tissue refers to a method of measuring or observing a tissue at set intervals or specific times, rather than constantly or in real-time (refers herein as continuous).

In some embodiments, employing the method of the present disclosure at least two of the temporarily time points allow a comparison between two states of the tissue and specifically of the at least one feature of the identified blood vessels as determined at different depths within the tissue.

In accordance with the present disclosure, the change that occurs between the two time points may be significant or relatively minor, but the comparison between them may provide insight into changes/developments in 3D blood vessels network over time.

The temporarily separated time points, either providing continuous monitoring or intermittent monitoring, may be any amount of time apart suitable to obtain characterization of blood vessels.

For example, the temporarily separated time points may be a few seconds, minutes, hours, days and even more.

In some embodiments, the gap between the two temporarily separated time points is about 1 millisecond, about 5 milliseconds, about 10 milliseconds, about 50 milliseconds, about 100 milliseconds, about 500 milliseconds.

In some embodiments, the gap between the two temporarily separated time points is about 1 second, about 5 seconds, about 10 second, about 50 second.

In some embodiments, the gap between the two temporarily separated time points is about 1 minute, about 5 minutes, about 10 minutes, about 50 minutes.

In some embodiments, the gap between the two temporarily separated time points is about 1 hour, about 5 hours, about 10 hours, about 24 hours.

In some embodiments, the gap between the two temporarily separated time points is about 1 day, about 5 days, about 10 days, about 14 days.

In some embodiments, the gap between the two temporarily separated time points is about 1 month, about 5 months.

The present disclosure is not limited to a specific tissue and is applicable to various tissues in a subject.

In some embodiments, the tissue is associated with changes in blood vessels with time.

In some embodiments, the changes in blood vessels comprise destruction of blood vessels.

As used herein the term destruction of blood vessels refers to the process of damaging or breaking blood vessels, for example the walls of blood vessels. These processes may in turn result in bleeding and impaired blood flow to tissues and may occur in different conditions, such as trauma, disease, or medical procedures.

In some embodiments, destruction of blood vessels is associated with uterine fibroids, atherosclerosis, aneurysms, vasculitis, and hemorrhagic stroke. Hence, the methods described herein are applicable for monitoring (diagnosis) of one or more of uterine fibroids, atherosclerosis, aneurysms, vasculitis, and hemorrhagic stroke.

In some embodiments, the changes in blood vessels comprise enhanced growth of blood vessels in said tissue.

As used herein the term enhanced growth of blood vessels, encompasses a normal physiological process of blood vessels growth or a pathological process of blood vessels growth.

The enhanced growth may be abnormal growth of blood vessels leading to damage to the tissue.

As noted herein above, an example process of blood vessel growth is angiogenesis. While in some tissues, angiogenesis is essential for tissue growth and repair, as it allows for the delivery of nutrients and oxygen to tissues, in certain pathological conditions such as cancer, angiogenesis can become excessive, leading to the formation of abnormal blood vessels that can promote tumor growth and metastasis.

In some embodiments, the tissue comprises at least a portion of the blood vessels that can be viewed from outside the tissue.

In some embodiments, the tissue is selected from the group consisting of: liver, kidneys, lungs, brain, heart, intestine, muscles, skin, a retina, uterine and a cancerous tissue.

In some embodiments, the tissue is uterine.

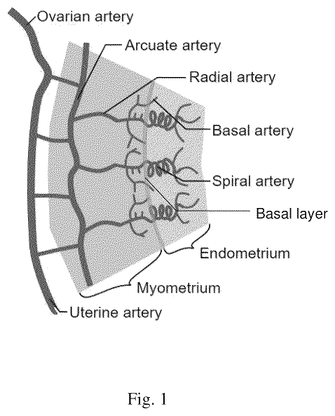

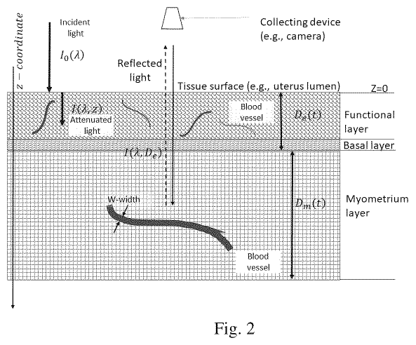

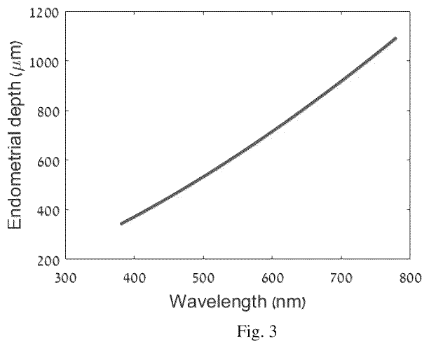

A schematic representation of the uterine is shown in FIG. 1. In addition, FIG. 2 shows a schematic representation of the uterine tissue comprising the endometrium tissue that is composed of a functional layer and a basal layer. As further shown in FIG. 3 representing a model-based graph, a visible light can penetrate the endometrium tissue and hence provide information on different depths within this tissue as discussed herein.

In some embodiments, the tissue is the endometrium tissue. Endometrium refers to the inner lining of the uterus. In the endometrium tissue, blood vessels distribution and evolution play a distinctive role in the tissue characterization. During the feminine menstrual cycle of healthy, fertile women (or mammals in general) the blood vessel three-dimensional network changes at a relatively short period of time. Most of the endometrium tissue is being generated and washed out during the cycle within the timescale of tens of days. Well accepted is the paradigm that angiogenesis within the endometrium is closely related to the success or failure of, e.g., embryo implantation.

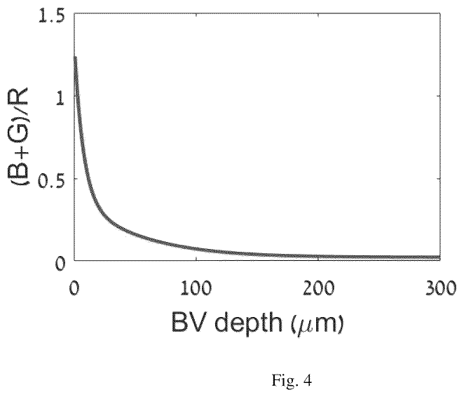

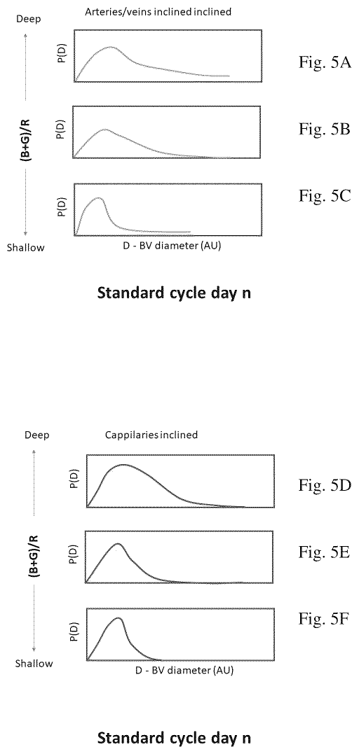

FIGS. 5A-5F show two different theoretical scenarios of blood vessels evolution, with FIGS. 5A-5C showing blood vessels evolution led primarily by elongation, followed by capillary growth through splitting angiogenesis. In this model, on the shallower strata (FIG. 5C) relatively big diameter blood vessels are found. FIGS. 5D-5F show a model by which blood vessels evolution of newly found layers is primarily governed by sprouting of newly formed capillaries and as such shallow strata (FIG. 5F) do not contain blood vessels with large diameter. It should be noted that in case time permits, blood vessels with large diameter may be formed at the deeper layers.

In humans, blood supply to and within the endometrium is maintained by a cascade of blood vessels of various diameters. The uterine artery, coming off the ovarian one, supplies blood to the arcuate arteries from which the radial arteries run radially towards the uterus lumen and split into the basal and spiral arteries (arterioles) which reside within the endometrium. The smallest diameter vessels in the endometrium undergo vascularization and form capillaries. This 3D blood vessel network promotes and controls the tissue growth, nourishes it and keeps up with its structure build-up. Blood vessels of smaller diameters emerge from the main arteries in ever-decreasing sizes and then, to the opposite direction reconnect with the main veins. All blood vessels within the endometrium are generated as vessels of small diameter and grow to their final diameter (before the endometrial shedding) in a controlled process.

As shown in FIGS. 8A-8F, FIGS. 9A-9F and FIGS. 12A-12E, a clear trend was observed between the blood vessel diameter and the depth.

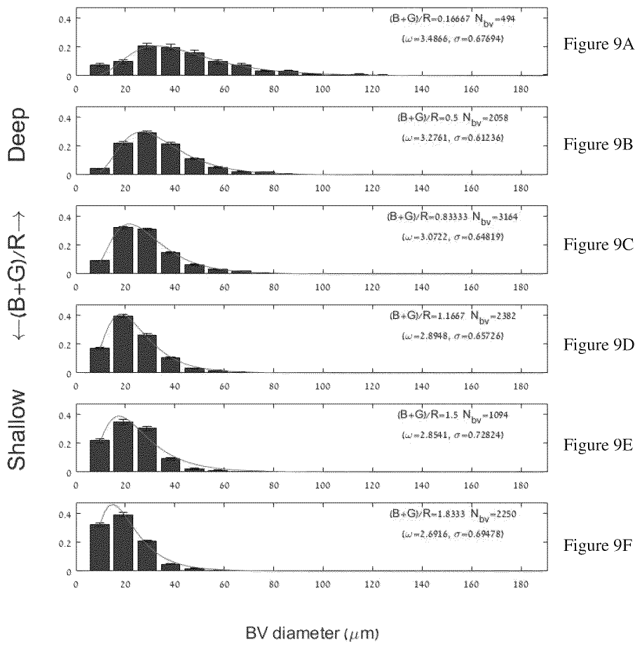

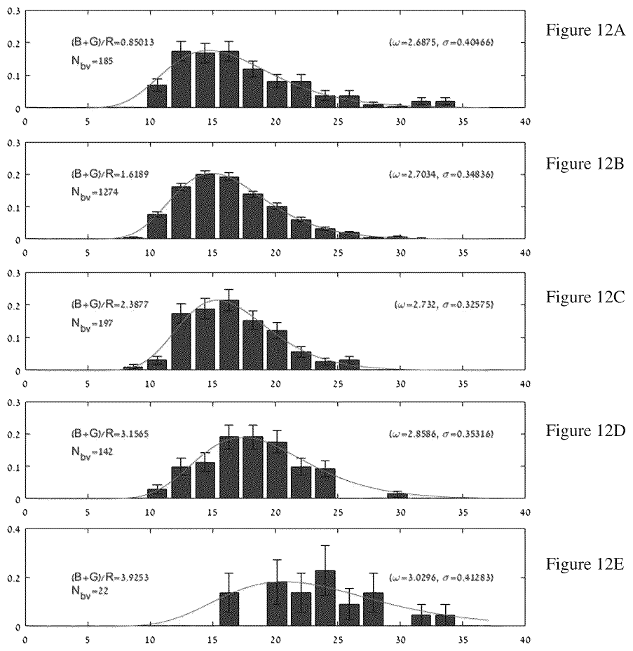

Interestingly, FIGS. 8A-8F and FIGS. 9A-9F providing data from human samples of the endometrium tissue, show a trend of a shortening distribution tails in blood vessels that are closer to the tissue surface. In contrast, FIGS. 12A-12E providing data from swine samples, a tail of relatively large diameter vessels was observed in the deeper layers. The difference in blood vessels as observed in the human samples vs. the non-human samples, suggest that the methods described herein may be considered as reliable method for following blood vessel evolution (such as changes in diameter) in various depth.

It was suggested that monitoring changes of the 3D blood vessels network in time and space (i.e., in different depths of the endometrium tissue), may provide information regarding the endometrium status and assists in evaluating the uterus readiness for e.g., embryo implantation.

Hence, in accordance with some aspects, the present disclosure provides a method for characterizing at least one feature of blood vessels at different depths within an endometrium tissue of a mammalian subject, the method comprising:
(i) identifying blood vessels in an image obtained from a surface of the uterine,
(ii) determining at least one feature of the identified blood vessels, a color index for the identified blood vessels or a combination thereof, wherein the color index differs in different depths within the endometrium tissue.

In accordance with some other aspects, the present disclosure provides a method for characterizing the diameter blood vessels at different depths within an endometrium tissue of a mammalian subject, the method comprising:
(i) identifying blood vessels in an image obtained from a surface of the uterine,
(ii) determining the diameter of the identified blood vessels, a color index for the identified blood vessels or a combination thereof, wherein the color index differs in different depths within the endometrium tissue.

As noted herein, the development of blood vessels in the uterus is a controlled process and hence it is valuable to characterize one or more features of the blood vessels, including, inter alia, the diameter blood vessels at different depths within an endometrium tissue of a mammalian subject in different time points.

In some embodiments, the method comprises repeating steps (i) and/or (ii) at a temporally separated time point as described herein.

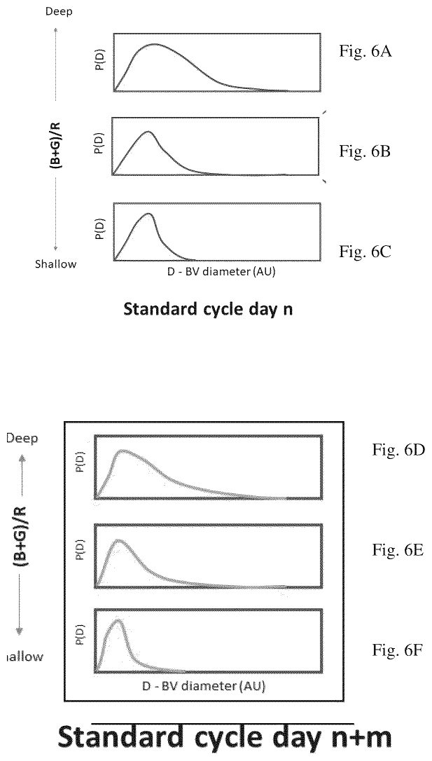

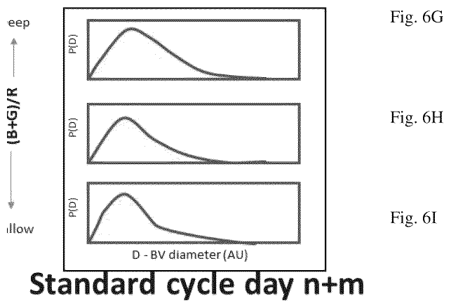

FIGS. 6A-6I show two different theoretical representations that may represent different individual subjects and their recorded blood vessels diameter PDFs as function of tissue depth. Both individuals have the same PDF on same day shown in FIGS. 6A-6C, yet after m days, it was possible to distinguish between two different patterns in the development of the blood vessels. Specifically, the blood vessels of the theoretical individual represented in FIGS. 6D-6F did not evolve to the degree they should in order to fit their calendar day, whereas the blood vessels of the theoretical individual represented in FIGS. 6G-6I, evolved at the standard pace to show the diameter PDFs as function of tissue depth, exactly the way they should be at the calendar time.

It was suggested that by obtaining information on the 3D blood vessel network in the uterine tissue and specifically in the endometrium tissue at different depths and different times points, it would be possible to contribute to precise endometrial dating and may assist in more accurate determination of embryo transfer timing within in vitro fertilization (IVF) treatments.

It was further suggested that by obtaining information on the 3D blood vessel network in the uterine tissue and specifically in the endometrium tissue at different times points, it would be possible to assess changes occurring in the uterus during the menstrual cycle. Such information may be valuable in connection with various clinical situations.

In some embodiments, the method is for monitoring menstrual cycle in a subject.

The menstrual cycle is a natural reproductive process occurring in females of reproductive age that involves a series of hormonal and physiological changes that prepare the uterus for pregnancy each month. The menstrual cycle typically lasts 28 days, but it can range from 21 to 35 days in some individuals. During the menstrual cycle, the body undergoes several phases, including the menstrual phase (when the uterus sheds its lining), the follicular phase (when the ovary prepares to release an egg), ovulation (when an egg is released from the ovary), and the luteal phase (when the uterus prepares for a possible pregnancy). These changes are accompanied by changes in the blood vessels, which supply the tissue with oxygen and nutrients.

It was suggested that monitoring changes in the 3D blood vessels network using the methods of the present disclosure may provide valuable information about the health of the endometrial tissue and help diagnose conditions such as endometrial hyperplasia, which can increase the risk of endometrial cancer.

Hence, the present disclosure provides in accordance with some aspects, a method for monitoring menstrual cycle in a subject, the method comprises: (i) identifying blood vessels in an image obtained from a surface of the uterine, (ii) determining at least one feature of the identified blood vessels, a color index for the identified blood vessels or a combination thereof, wherein the color index differs in different depths within the endometrium tissue.

In accordance with some embodiments, the method is for monitoring menstrual cycle in a subject and the at least one feature is the diameter of the identified blood vessels.

In accordance with some embodiments, the method is for monitoring menstrual cycle in a subject, the at least one feature is the diameter of the identified blood vessels and method comprises repeating steps (i) and/or (ii) at a temporally separated time point as described herein.

In some embodiments, the method is for diagnosis of infertility and/or infertility-related conditions in a subject.

It was suggested that monitoring changes in the 3D blood vessels network using the methods of the present disclosure may provide valuable information about infertility and/or infertility-related conditions.

Hence, the present disclosure provides in accordance with some aspects, a method for diagnosis of infertility and/or infertility-related conditions in a subject, the method comprises:
(i) identifying blood vessels in an image obtained from a surface of the uterine, (ii) determining at least one feature of the identified blood vessels, a color index for the identified blood vessels, or a combination thereof, wherein color index differs in different depths within the endometrium tissue.

In accordance with some embodiments, the method is for evaluating infertility infertility-related conditions in a subject and the at least one feature is the diameter of the identified blood vessels.

In accordance with some embodiments, the method is for evaluating infertility infertility-related conditions, the at least one feature is the diameter of the identified blood vessels and method comprises repeating steps (i) and/or (ii) at a temporally separated time point as described herein.

The term infertility-related conditions as used herein refers to any medical condition that may lead to difficulty in conceiving or carrying a pregnancy to term.

In some embodiments, the infertility-related conditions comprise one or more of the following: ovulation disorders, tubal blockage, endometriosis, uterine or cervical abnormalities or age-related infertility.

In some embodiments, the method is for selecting the timing of embryo transfer and implantation in a subject.

Hence, the present disclosure provides in accordance with some aspects, a method for selecting the timing of embryo transfer and implantation in a subject in a subject, the method comprises: (i) identifying blood vessels in an image obtained from a surface of the uterine, (ii) determining at least one feature of the identified blood vessels, a color index for the identified blood vessels, or a combination thereof, wherein the color index differs in different depths within the endometrium tissue.

In accordance with some embodiments, the method is for selecting the timing of embryo transfer and implantation in a subject in a subject and the at least one feature is the diameter of the identified blood vessels.

In accordance with some embodiments, the method is for selecting the timing of embryo transfer and implantation in a subject, the at least one feature is the diameter of the identified blood vessels and method comprises repeating steps (i) and/or (ii) at a temporally separated time point as described herein.

In some embodiments, the method is for evaluating receptivity of the endometrium to embryo implantation in a subject.

Hence, the present disclosure provides in accordance with some aspects, a method for evaluating receptivity of the endometrium to embryo implantation in a subject in a subject, the method comprises: (i) identifying blood vessels in an image obtained from a surface of the uterine, (ii) determining at least one feature of the identified blood vessels, a color index for the identified blood vessels, or a combination thereof, wherein the color index differs in different depths within the endometrium tissue.

In accordance with some embodiments, the method is for evaluating receptivity of the endometrium to embryo implantation in a subject in a subject and the at least one feature is the diameter of the identified blood vessels.

In accordance with some embodiments, the method is for evaluating receptivity of the endometrium to embryo implantation in a subject the at least one feature is the diameter of the identified blood vessels and method comprises repeating steps (i) and/or (ii) at a temporally separated time point as described herein.

In some embodiments, the method is for determining suitability for embryo transfer and implantation in the uterine of a subject.

It was suggested that monitoring changes in the 3D blood vessels network using the methods of the present disclosure may provide valuable information about timing of embryo transfer and implantation in a subject, the receptivity of the endometrium to embryo implantation in a subject and/or suitability for embryo transfer and implantation in a uterine of a subject.

Hence, the present disclosure provides in accordance with some aspects, a method for evaluating suitability and timing of embryo transfer and implantation in a subject and/or receptivity of the endometrium to embryo implantation in a subject, the method comprises (i) identifying blood vessels in an image obtained from a surface of the uterine, (ii) determining at least one feature of the identified blood vessels, a color index for the identified blood vessels, or a combination thereof, wherein the color index differs in different depths within the endometrium tissue.

In accordance with some embodiments, the method is for evaluating suitability and timing of embryo transfer and implantation in a subject and/or receptivity of the endometrium to embryo implantation in a subject and the at least one feature is the diameter of the identified blood vessels.

In accordance with some embodiments, the method is for evaluating timing of embryo transfer and implantation in a subject and/or receptivity of the endometrium to embryo implantation in a subject, the at least one feature is the diameter of the identified blood vessels and method comprises repeating steps (i) and/or (ii) at a temporally separated time point as described herein.

In some embodiments, the subject is a female subject. In some embodiments, the subject is considered to undergo or in the process of IVF.

In some embodiments, in which the method employs repeating method steps at a temporally separated time point, the two times points are selected between day 14 and day 22 of the cycle, wherein the cycle is normalized to produce a 28 days cycle.

As shown in the Examples below, the inventors used images of fresh, ex-vivo, endometrial samples of different cycle days to obtain the statistical evolution track of the blood vessel population in both human and animal (swine) samples.

In some embodiments, the method is an ex-vivo method.

In some embodiments, the method is an in vivo method.

For the purpose of diagnosis, the at least one feature and/or any change of the at least one feature over time may be compared to the same feature and/or the change of the at least one feature over time in a fertile female. Fertile female as used herein refers to a woman who has the physical ability to conceive and carry a pregnancy to term.

As noted herein, the methods described herein are applicable for a variety of tissue and hence may be applicable for determining a pathological condition of a subject.

It was suggested that monitoring changes in the 3D blood vessels network using the methods of the present disclosure may provide valuable information about a pathological condition of the subject.

Hence, the present disclosure provides in accordance with some aspects, a method for determining a pathological condition of a subject in a subject, the method comprises:
(i) identifying blood vessels in an image obtained from a surface of a tissue, (ii) determining at least one feature of the identified blood vessels, a color index for the identified blood vessels, or a combination thereof, wherein the color index differs in different depths within the tissue. In some embodiments, the method comprises repeating steps (i) and/or (ii) at a temporally separated time point as described herein.

In accordance with some embodiments, the method is for determining a pathological condition of a subject and the at least one feature is the diameter of the identified blood vessels.

In some embodiments, the method is for determining a pathological condition of a subject, the at least one feature is the diameter of the identified blood vessels and the subject is diagnosed with a condition associated with enhanced growth of blood vessels in the tissue. In some other embodiments, the subject is suffering from at least one proliferative disorder.

As used herein, “proliferative disorder” is a disorder displaying hyper proliferation. This term means cell division and growth that is not part of normal cellular turnover, metabolism, growth, or propagation of the whole organism. Unwanted proliferation of cells is seen in tumors and other pathological proliferation of cells, does not serve normal function, and for the most part will continue unbridled at a growth rate exceeding that of cells of a normal tissue in the absence of outside intervention. A pathological state that ensues because of the unwanted proliferation of cells is referred herein as a “hyper proliferative disease” or “hyper proliferative disorder.” It should be noted that the term “proliferative disorder”, “cancer”, “tumor” and “malignancy” all relate equivalently to a hyperplasia of a tissue or organ.

The present dasyure also provides in accordance with some other aspect, a method for diagnosing a pathological condition in a subject, the method comprising (a) determining at least one feature characteristics of blood vessel population from one or more images obtained from a tissue of said subject, said one or more images is obtained by light imaging and (b) determining if the subject is suffering from said pathological disorder, wherein said pathological condition associated with enhanced growth and/or formation of blood vessels in said tissue.

The present dasyure also provides in accordance with some other aspect, a method of assessing responsiveness to a treatment regimen for a subject suffering from a pathological disorder and monitoring disease progression of said subject, the method comprising: (a) determining at least one feature characteristics of blood vessel population from one or more images obtained from said subject, said one or more images is obtained by visible light imaging and (b) determining if the subject is responsive or non-responsive to the treatment regimen.

As described herein, the methods comprising repairing the steps of determining at least one feature characteristics of blood vessel population from one or more images obtained from a tissue of said subject in at least one temporarily separated time point. In some examples, the method comprises comparing the at least one feature in the at least two temporarily separated time point.

The present invention relates to subjects, individuals or patients. By “patient”, “individual”, “individuals “or” subject” it means any organism who may be affected by the above-mentioned conditions, and to whom methods herein described is desired, including humans. More specifically, the methods of the invention are intended for mammals. By “mammalian subject” is meant any mammal for which the proposed therapy is desired, including human, equine, canine, and feline subjects, most specifically humans and more specifically a female.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

It should be noted that various embodiments of this invention may be presented in a range format. The description of a range should be considered to have specifically disclosed all the possible sub ranges as well as individual numerical values within that range. For example, description of a range such as from 1 to 6 or between 1 and 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

As used herein, the forms “a”, “an” and “the” include singular as well as plural references unless the context clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments unless the embodiment is inoperative without those elements.

It should be noted that the various embodiments and examples detailed herein in connection with various aspects of the invention may be applicable to one or more aspects disclosed herein. It should be further noted that any embodiment described herein, for example, related to method, may be applied separately or in various combinations. Various embodiments and aspects of the present invention as delineated hereinabove and as claimed in the claims section below find experimental support in the following examples. The phrases “in another embodiment” or any reference made to embodiment as used herein do not necessarily refer to different embodiment, although it may. Thus, various embodiments of the invention can be combined (from the same or from different aspects) without departing from the scope of the invention.

Various embodiments and aspects of the present invention as delineated herein above and as claimed in the claims section below find experimental support in the following examples.

Disclosed and described, it is to be understood that this invention is not limited to the particular examples, methods steps disclosed herein as such methods steps may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### NON-LIMITING EXAMPLES

### Materials and Methods

### Human Clinical Trial

Forty-nine samples of endometrial tissue were collected from 37 recurrent implantation failure (RIF) patients that were about to undergo in vitro fertilization (IVF) treatments in the subsequent cycle.

The key inclusion criteria for the participants were (i) IVF patients diagnosed with RIF who were regularly ovulating; (ii) Age: 18-40 years and (iii) both patients whose fertility status was unknown and patients who had proven to be fertile (previous successful pregnancy).

Key exclusion criteria were (i) patients with known existing endometrial pathology, (ii) patients with a known history of infertility due to oligo-ovulation or anovulation, (iii) patients with a medical history of malignant tumors in their reproductive system, (iv) patients that were on any hormonal medications or hormonal treatment (excluding hormonal contraception in previous cycles), (v) patients who were on hormonal contraception treatment in their current cycle, (vi) patients carrying IUD and (vii) patients menstruating on the day of the biopsy collection.

For each participant the cycle dating was performed utilizing the following four methods:

### 1. Testimony

Participant's report of the first day of her last menstrual cycle, her average cycle and menses duration, and the variance in both.

### 2. Histology

Histopathological evaluation by two expert Gyneco-histopathologists. The two had to reach consensus. This is commonly regarded as the gold standard method for cycle dating.

### 3. Hormone Levels

Blood sample for hormones level analysis (LH, FSH, ER, PR, Access 2 analyzer (Beckman & Coulter); Advia Centaur XP (Siemens).

### 4. Ultrasound

Endometrial morphology and thickness measured by vaginal ultrasound examination.

All methods were normalized to produce a 28 day “standard” cycle, using the patient report or the hormone manufacturer (i.e., Abbott) data.

The fresh ex-vivo samples (<3 hours post collection) were examined under a stereoscope (Motic SMZ-171-TL) equipped with a ring 144-Led white light illumination source and a mounted camera (Motic MotiCam3). Images at 2× and 4× magnifications of endometrial surface areas were captured.

A proprietary dedicated image analysis software (under Matlab Ver. R2016a of MathWorks) identified blood vessels on the image of the tissue surface samples and ascribed attributes (e.g., average diameter, average color, etc.) to each identified blood vessel. Specifically, an average value was calculated for R (red), G (green) and B (blue) for all pixels in a blood vessel, to obtain a R_avg, G_avg, B_avg for each modeled blood vessel. Taken together, for each modeled blood vessel the average diameter (denoted herein as “w”) was calculated together with the average R value, average G value and average B value.

### Swine Pre-Clinical Trial

Four female swine (Age=1.8±0.6 years, Body mass=215±25 kg) were designated from an animal research institute (“Lahav CRO”, Israel). Key inclusion criteria were: (i) healthy, proven-fertile, non-pregnant female swine; (ii) age 1-4 years; (iii) not on any medication in the two months prior to selection.

Exclusion criteria included: (i) irregular diet, (ii) harmed or damaged uterus or menstruation during the surgical experiment. The study, using common laboratory animals, was approved by the Israeli National Ethics Committee (#IL-14-11-292).

The average length of a regular menstrual cycle in the sow is 21 days ((Martinat-Botté et al., 2000)). Cycle day is expressed from day 0 to day 20. Here, day 0 was defined as the beginning of the LH surge.

The cycle day for each one of the sows was determined using three different conventional methods:

### 1. Behavioral Analysis

Estrus date (˜cycle day 2) was determined by an experienced veterinarian based on standing heat and standard behavioral characteristics of the female sow and that of a male swine in its proximity.

### 2. Hormone Levels

Three blood samples were drawn from the sows: one sample on the selection date, two others on the surgical experiment day, approximately halfway between the two dates. Blood samples were analyzed (AML Israel, Ltd., Herzliya, Israel) for Estradiol and Progesterone levels. Progesterone levels were obtained from two machines [Cobas e601 (Roche Diagnostics) and Immulite 2000 (Siemens) for the latter, due to one uncertain measurement on the Cobas machine]. Cycle day was determined by a two-term Chi-square analysis.

### 3. Histology

Following ex-vivo imaging of the endometrium, a biopsy was extracted from each imaged uterine location. For each biopsy, 5 μm histology H&E slides were prepared with several longitudinal sections (L.E.M. Ltd.). Pathology analysis was performed in Pharmaseed Ltd. (both: Nes Tziona, Israel) by an expert in veterinary pathology, in consultation with a world-renowned expert in swine histology and endometrium dating.

### Analysis Method

A schematic view of the endometrium and the tissues abut it, along with the basic morphology of the main arteries cascade is shown in FIG. 1 showing, for example, the endometrium and the myometrium. As can be seen from the schematic representation, the endometrium is made of two morphologically undistinguishable layers: the functional layer (functionalis) and the basal layer below it which resides on top of the myometrium.

The different layers and coordinates are schematically shown in FIG. 2.

The depth coordinates are depicted as the distance from the endometrium surface level facing the uterus lumen, by the coordinate z.

Typical z-dimensions for the abovementioned tissue strata are 1-2 mm for the basal layer and 0-6 mm for the functional layer. The z-dimension of the functionalis grows and sheds regularly in the course of the feminine cycle.

The x- and y-coordinates are taken to be the (arbitrarily oriented) coordinates as registered on a planar image of the endometrium surface, taken from the lumen side.

An infinitesimal volume element ΔV, is thus defined as ΔV=ΔxΔyΔz.

Assuming that De (t) be the endometrium total depth (functional & basal layer together) as function of time, t; Dₘ(t) the myometrium total depth as function of time and Dᵤₑ(t)=Dₘ(t)+Dᵤ(t) the entire uterus tissue depth as function of time (as shown in FIG. 2).

Recording blood vessels (BV), may depend on various attributes. Among these attributes one may list, non-exhaustingly, the blood vessel average (or min/max) diameter, the blood vessel average length, or its length between bifurcations or splits, the blood vessel degree of tortuosity, the oxygen saturation level within the blood vessel, the flow pattern and the flow magnitude profiles within the blood vessel, etc.

For each one of these attributes, «i, one may construct a probability distribution function (PDF) that provides one of two probability density functions ([0-1] range):
1. The absolute probability of finding a BV within the tissue volume ΔV, that possesses the attribute o within the range [∝ᵢ′, ∝ᵢ′+Δ∝ᵢ′] denoted by P(BV, ∝ᵢ)
2. The conditional probability for a given BV to have its attribute ∝ᵢ within the range [∝ᵢ′+∝ᵢ′] denoted by P(∝ᵢ|BV).

The first PDF is naturally the multiplication of the second PDF by the absolute probability of finding a BV within the volume ΔV. This latter probability is non-homogeneous within the endometrium nor it is constant in time. It should therefore be actually defined as P(BV)≡P_{BV}=P(z,t), where specific homogeneity in x-y (at a large enough ROI and specific z and t) is assumed and the probability is derived from the BV number density as function of depth and time within the infinitesimal tissue volume ΔV.

One may also have such PDFs for more than a single attribute, either as free probability or conditional, i.e., one of the following combinations:
a. P(BV, α₁, . . . , αₙ)
b. P(α₁, . . . , αₙ|BV)
c. P(αⱼ, . . . , αₙ|BV, αᵢ, . . . , αₘ), where the two sets of attributes are strange to each other

In order to demonstrate the proposed method, focus was made on a specific attribute ∝, which is the width of the BV, w, as measured on the tissue image.

Since the diameter may change along the BV length, the “average width of the recorded BV along its length” is referred as the “BV diameter” or, width.

Hence, w is defined to be the average width of a blood vessel of length l (on the image). Alternatively, it is possible to use the maximum value of the width or any other attribute thereof. For the average width we have

$$w = \frac{1}{l} {\int }_{0}^{l} {w}^{′} ( {l}^{′} ) {dl}^{′}$$

Assuming a circular BV cross-section the average width can be readily translated to the average diameter of the BV in spite of the projection effects of the image and regardless of the BV three-dimensional orientation with respect to the image plane.

Similarly, the BV z-coordinate may change along the BV length, and thus can be refer to its average z-coordinate.

At a specific time, t, and an arbitrary depth, z, assume a PDF of the BVs width, w. A good exemplary choice of such PDF functional form could be a Log-normal distribution, i.e., for the variable, P(w, z|BV), the probability density of a given BV to be of width between w and w+Δw is expressed via the log of w, ω=log(w) to be

$$P ( w , z \vert B V ) = \frac{1}{w} \frac{1}{σ \sqrt{2 π}} exp ( - \frac{{( ω - \overline{ω} )}^{2}}{2 {σ}^{2}} ) ,$$

which is a Log-normal distribution with ω̅=ω̅(z) being the mean value of log(w) and σ=σ(z) the standard deviation of log(w).

The intensity of the incident light (of amplitude: I(z=0, 1)≡I₀(λ)) at depth z below the lumen (within the endometrium, namely for z<Dₑ) experiences attenuation according to the equation I(z, λ)=I₀(λ)e^{-μ}^{ₑ}^{(λ)z}, where μ(λ)ₑ is taken to be the total (effective) attenuation coefficient, considering both absorption and scattering, including the anisotropy factor, g.

The absorption coefficient of tissues varies significantly in the visible spectrum, whereas the scattering coefficient decreases monotonically as the wavelength increases. The latter can be modeled as a combined contribution of Rayleigh and Mie scattering and is mostly responsible for the relative higher penetrability of longer wavelengths (“red vs. blue”) in the visible spectrum.

A good approximation to the scattering dependence on wavelength is given by

$${μ}_{e} ( λ ) \approx f {λ}^{- 4} + ( 1 - f ) {λ}^{- b} ,$$

Where f is the relative fraction between the Rayleigh expression (left) for scattering contribution due to particles of size much smaller than the light wavelength and the Mie scattering (right expression) for scatter by particles comparable in size.

Jacques, 2013 provides a useful approximation for the total extinction coefficient, along with a compilation of experimental values for a′, f and b for various tissues.

$${μ}_{e} ( λ ) \approx {a}^{′} ( {f ( \frac{λ}{500 ( nm )} )}^{- 4} + ( 1 - f ) {( \frac{λ}{500 ( nm )} )}^{- b} ) ,$$

At the transition endometrium-myometrium I(De, λ)=I₀(λ)e^{-μ}^{ₑ}^{(λ)D}^{ₑ} and for a blood vessel located at depth z>Dₑ below the lumen, I(z>Dₑ, λ)=I₀(λ)e^{-μ}^{ₑ}^{(λ)D}^{ₑ}e^{-μ}^{ₘ}^{(λ)(z-D}^{ₑ}⁾. We do not expect visible light penetration beyond the myometrium, though other wavelengths may reach deeper. The formalism we present herein should not be limited to any depth or tissue structure.

For blood reflection coefficient, [symbol] (λ), (assuming tissue refractive index ˜1 here), the intensity of the reflected light, after reflecting from a blood vessel located at z_{BV}, is

$$I ( {z}_{B V} > {D}_{e} , λ ) = {I}_{0} ( λ ) {e}^{- {μ}_{e} ( λ ) {D}_{e}} {e}^{- {μ}_{m} ( λ ) ( {z}_{B V} - {D}_{e} )} ℛ ( λ )$$

And after its journey back it becomes

$${I}_{BV} ( λ ) = \{ \begin{matrix}{I}_{0} ( λ ) {e}^{- 2 {μ}_{e} ( λ ) {D}_{e}} {e}^{- 2 {μ}_{m} ( λ ) {z}_{BV} - {D}_{e} )} ℛ ( λ ) & {z}_{BV} > {D}_{e} \\ {I}_{0} ( λ ) {e}^{- 2 {μ}_{e} ( λ ) {z}_{BV}} ℛ ( λ ) & else\end{matrix}$$

It was assumed that blood itself is responsible for most of the reflection with a minor, negligible contribution from the vessel tissue itself. This has been justified by measurements of the blood spectrum in-vivo through its carrying vessels. Under extreme circumstances (e.g., pathological vessels), this assumption can be replaced by a linear combination of the reflection of the blood and the vessel.

FIG. 2 also shows a representative system for collecting data, such that a collecting device (for example a camera) captures the upper layer of the endometrium from within the uterus lumen. As shown in FIG. 2, there is a source of illumination which provide the incident light of a known spectrum from the same direction (but possibly at a different incident angle).

The z-coordinate is measured from the tissue surface inwards (i.e. down the z axis). The light first crosses the tissue surface, at a distance De it crosses the junction between the endometrium and the myometrium. The inner division between the endometrium functional and basal layers in unnoticeable by the light. The light may hit a blood vessel from that reflects it backwards (to the negative z-direction). The color ratio of the backscattered outcoming light is dictated by the tissue, the target (BV) depth (z-coordinate) and the reflection index of the vessel and mostly the blood.

As noted above, the endometrium is made of two layers: functional layer (sheds every menses) and basal layer (thin, survives the menses). The layers cannot be distinguished by regular histology and therefore, for optical practical purposes, it was approximated that the endometrium forms a single layer. Since the basal layer is between 1 to 2.5 mm thick [4], in the following demonstration, only the endometrium will be referred and hence neglecting the myometrium as visible light will not get to it.

As shown in FIG. 3, the penetration depth of visible light through an endometrium tissue justified the assumption that visible light will not get to the myometrium.

For a blood vessel of width w there is a maximum z beyond which it can no longer be detected due to the light low (attenuated) intensity, the noise level and the spatial resolution of the imaging device. To a first approximation, the wavelength intensity dependence was neglected, and reference was made to the peak of the blood reflection spectrum only. Also the spatial resolution dependence was neglected and a sharp width cutoff was applied instead (the minimal blood vessel diameter to be detected, regardless of its z coordinate.). This can be justified post fact after thin (small w) blood vessels are found at the deepest possible layer side by side with large diameter vessels. The z cutoff is taken to be

$${z}_{cutoff} = \{ \begin{matrix}\frac{2 - {D}_{e} ( {μ}_{e} - {μ}_{m} )}{{μ}_{m}} & z > {D}_{e} \\ \frac{1}{{μ}_{e}} & else\end{matrix}$$

which is equivalent to attenuation of the order ˜e⁻².

Taking into account the optical parameters (attenuation coefficients) of endometrium-related tissues, e.g., mucous tissue [5] [3] with a′=18.8 cm⁻¹; f=0.0; b=1.62 and a flat spectrum incident light (“white light”), this z_{cutoff} turns out to be at the range of 400-800 μm depending on the wavelength. FIG. 3 shows such wavelength dependence, where a factor ˜4 4 exists between the Blue light penetration depth and the Red one.

Next, a model for the BV width distribution function evolution in time was developed.

For the endometrium, in particular for its functional layer, there is not enough time to grow BVs beyond capillaries during the cycle (of up to 30 days). There is thus a very sharp descend of the distribution function toward the capillary upper diameter value, as long as the functional layer is considered.

Since arterioles and venules have at most w˜100 μm, and most capillaries are at w˜5-10 μm, these values determine the two log-normal parameters: ω̅, σ of the BV distribution within the endometrium, with a slight increase toward the strata bassalis that may, or may not be significant in the distribution alteration.

The myometrium, on the other hand includes BVs that are much larger than the ˜100μ upper limit and its population remains relatively constant throughout the cycle, namely the distribution time-dependence is much weaker than the one of the endometrium. However, due to its location below the basal layer and the z_{cutoff} layer, visual light won't get reflected off of BVs residing in it. Other light bands (IR) should be exercised for that matter at the expense of lower spatial resolution.

For evaluating the endometrium evolution, one would relate the z (depth) coordinate of a strata to its formation time, namely to its “age”. This should provide information with regards to the endometrial tissue evolution pace and its maturity, as reflected by the BV PDF, in comparison to its “calendar” age.

Assuming A(z) denote the “Age” of the endometrium layer at depth [z, z+Δz] measured from the lumen wall surface where z=0. If Dₑ(t) is known then for a given t we obtain A(z)=A (Dₑ(t))−A(Dₑ(t)−z).

If the BVs width distribution function is a function of the age of the endometrium tissue layer, obtain P(w|BV, z) may be obtained by ascribing the relation between z and A(z).

A sharp transition of the P(w|BV, z) is expected at the borderline where z=Dₑ, since the myometrium BV population is much more mature.

In order to utilize this PDF evolution formalism, one needs to estimate the z-coordinate of a given image-identified BV. To that end, it was proposed to use the statistical change in broadband colors of the blood vessels, as obtained due to the tissue differential attenuation in wavelength.

Since longer wavelengths are less attenuated than short wavelength (in the visual spectrum), one may use, e.g., the standard RGB camera channels to calculate broadband ratios such as

$$\frac{B}{R} or \frac{( B + G )}{R} .$$

These ratios change mainly due to the Mie scattering by the tissue. Even though conclusion regarding the BV depth coordinate for individual BV, based on that ratio is not trivial, the overall effect for the entire detected BV population can be clearly ascertained.

Blood mainly reflects in the long wavelengths and mostly contributes to the Red channel and thus the mere BV detection is feasible even at relatively large z-coordinate.

FIG. 4 shows the actual calculation of one of these color ratios. The BV reflection coefficients, [symbol] (1), are calculated based on the complementary values of the well-documented oxygenated blood absorption spectrum. Since broadband filters was used, the Oxy-Deoxy blood ratio is negligible in such calculation. Notice that at about 200 μm, the (G+B)/R ratio levels and does not allow differentiations between BVs residing deeper than this value. Other (e.g. IR) wavelength may somewhat alleviate this limitation. The ratios may contain information at deeper layers than 200 μm for more accurate transmission coefficient of the endometrium. Here the parameters from general epithelial or mucous layers were taken, but the endometrium, due to its relative transparency, may bear ratio information down to 400-500 μm. These depths are indeed smaller than the basal layer but provide detection of older (deeper) z-layers.

FIGS. 5A and 5B show schematic sets of BV diameter PDFs as function of depth for two competing evolution models (tracks) of BVs. The two columns represent tissues on the same standard cycle day that obeyed different evolution models. FIG. 5A, BV evolution is led primarily by elongation, followed by capillary growth through splitting angiogenesis. Therefore, even on the shallower strata (lowest panel) relatively big diameter BVs are found. FIG. 5B, show that shallow strata do not contain big diameter BVs, the BV evolution on newly found layers is primarily governed by sprouting of newly formed capillaries. In the deeper layer, where time permits, bigger diameter BVs may be found.

Uteri may differ in the three “clocks” they exhibit: the calendar time, the “standard” or “average” thickness versus time function and the function of the BV PDF versus z. This latter dependence should also have a “standard” (namely average over the population) behavior and deviations from this average may hint or relate to deviations in the other two “clocks”.

FIGS. 6A-6I show two different theoretical representations that may represent different individual subjects and their recorded blood vessels diameter PDFs as function of tissue depth. Schematically, both individuals are characterized by the same PDF (FIGS. 6A-6C) and started from the same day “n” of their standardized cycle day.

However, “m” days post the starting point of day “n”, the PDF of the individual represented in FIGS. 6D-6F did not evolve to the degree they should in order to fit their calendar day. The PDF of the individual represented in FIGS. 6G-6I, evolved at the standard pace to show the diameter PDFs as function of tissue depth, exactly the way they should be at the calendar time.

For example, blood vessels PDF that does not evolve as rapidly as expected, in spite of thickness growth, may allude to a “retarded” uterus that shall become ready for e.g., implantation at a later-than-“standard” time as shown for example in FIGS. 6D-6F.

The blood vessel network within the endometrial surface images was statistically analyzed the, using all of the sample's acquired images (2× and 4× separately), by treating the BV population as a statistical ensemble. An exemplifying image is shown in FIGS. 7A and 7B, where blood vessel computerized identification is overlaid (fully as shown in FIG. 7A and partially as shown in FIG. 7B). As such, probability distribution functions (PDFs) and the cumulative PDFs (cPDFs) were derived to describe the BV population.

The focus was on two BV features:
(i) w—the of the BV average width (along its length), which is equivalent to its diameter
(ii) the BV average color ratio, as calculated from its image Red-Green-Blue (RGB) colors.

The color ratio was used:

$$ℛ = \frac{( B + G )}{R} .$$

This ratio, [symbol] , is considered to represent z—the BV average depth beneath the tissue surface that faces the lumen of the uterus. Such representation is based on differential absorption and scattering models for different light wavebands (Bashkatov et al., 2011; Jacques, 2013).

The PDFs of the BV's diameter and color ratio may be functions of the cycle dating, t, or conditional probability such as the diameter distribution given a certain tissue depth (color ratio). These functions may or may not eliminate the normalization factor of the probability of finding a BV (of any diameter) within the unit volume ΔV( [symbol] ) of tissue surface unit area ΔS.

The BV PDF was modeled at a specific time and depth (t and z) with a Log-normal distribution with the mean (log) of its diameter ω̅=ω̅(z) and its standard deviation σ=σ(z).

### Results

### Human Samples

FIGS. 8A-8F depict the blood vessel diameter distribution function, as calculated from a sample of 344 2× magnification images. All images of all patients, regardless of their cycle day, were collected together to yield the general behavior or blood vessel diameter distribution differences as a function of tissue depth.

The deepest tissue layer (smallest color ratio, [symbol] , denoted herein as color index) is shown on FIG. 8A having a [symbol] (z) value of 0.16667 and a blood vessel diameter of 3.5772 (w), deeper layers by the order of their [symbol] , bin values, are shown on FIG. 8B to FIG. 8E. FIG. 8F shows the most superficial tissue layer which abuts the uterine cavity having a [symbol] (z) value of 1.833 and a blood vessel diameter of 3.0863 (w).

The raw histograms of the BV diameter distribution, as drawn from the total number of identified BVs in the layer's depth (N_{BV}) are also shown. The solid curve line is the overlaid log-normal function model of P(w| [symbol] ᵢ< [symbol] ≤ [symbol] ᵢ₊₁) and its corresponding fitting parameters (ω, σ), as well as the average layer's value ( [symbol] bin average value).

FIGS. 9A-9F show the results of the analysis as obtained from 625 images taken under 4× magnification, where the [symbol] bin limits and average values within each depth bin (layer) are identical to those in the 2× figure (FIGS. 8A-8F).

In both FIGS. 8A-8F and FIGS. 9A-9F a clear trend of increased w (“average diameter”) as function of layer's depth exists, whereas the change in the distribution “width” (o) as function of depth is less evident due to the image resolution, i.e., the narrowest identifiable BV. Specifically, as can be seen from FIGS. 8A-8F, the average diameter was the highest in the deeper layer (FIG. 8λ).

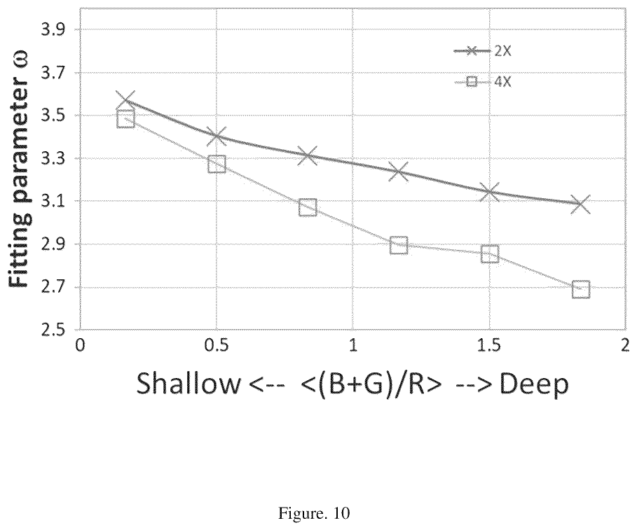

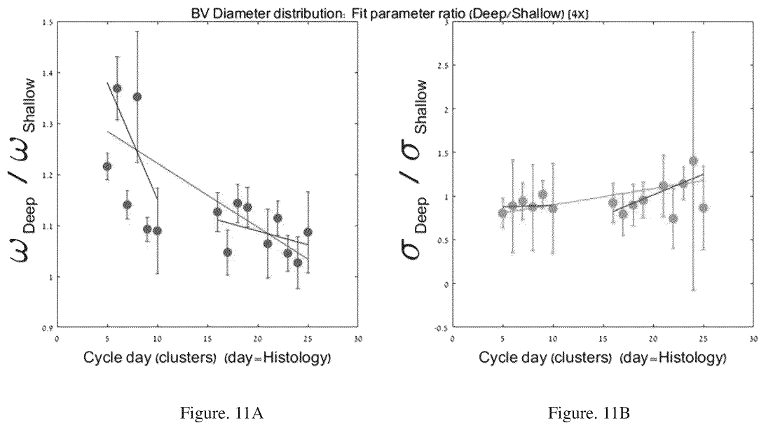

FIG. 10 shows the change in the w fitting parameter of the PDF for the BV diameter distribution as a function of the tissue layer's depth, namely, by interpretation, its distance from the lumen of the uterine cavity. The independent results from the 2× magnification sample (X symbol) and 4× (square symbols) are also shown. The w parameter is in the log and hence the small values on the y-axis. The difference in the narrowest identifiable BV, due to the higher spatial resolution under the 4× magnification, causes the sharper descent of the w values a there is an increase in the layers under the 4× magnification. Following our realization of a distinct global trend for BV diameter distribution as function of the endometrial layer, we turned to time-evolution of the BV diameter-color relationship. When assessed individually, each one of the two features (diameter, color) ensemble distribution, did not show clear evidence for an evolution track along the cycle time course. However, the combination of the two characteristics, namely the conditional probability does clearly display an evolution line. We split the color space into two bins only, as opposed to the six color bins in FIGS. 8A-8F and FIGS. 9A-9F. This was done in order to reduce sampling errors due to the smaller number of captured images for each cycle day.

All patient images of the same cycle day (as determined by histology) were collated together to obtain the BV diameter PDFs within the two-color bins. Each PDF was then modelled by the log-normal distribution and fitting parameters were derived along with their confidence level. For each one of the two fitting parameters (ω, σ) we plotted the ratio of the parameter as calculated by the deep half of the tissue (at that cycle day) versus the superficial half. FIG. 11A depicts the results of these ratios as a function of the cycle day such that the ratio of the distribution “average” (w in the log space), shows a clearcut evolution line. The global regression line (long line) does not adequately describe the ratio evolution in time, but the two regression lines, one for each cycle phase, provides a good modelling of the parameter ratio evolution. Interestingly, the slope change of the ratio evolution shows a pivotal point at or near day 14 (“ovulation”), which separated the two cycle phases.

The same slope change appears on FIG. 11B, where the ratio of the “width” (σ, in log) fitting parameter evolution is depicted.

It is important to note that the parameter ratio within the two-color bins, is rather constant throughout day 14, and thereafter it changes its slope. In general, as the endometrium progresses through the secretory phase, the BV diameter distribution of the superficial layer is narrower (“tighter”) that that of the deep layer.

### Swine Samples

All samples taken together, regardless of the cycle day yielded the color (depth)—diameter distribution as depicted in FIGS. 12A-12E.

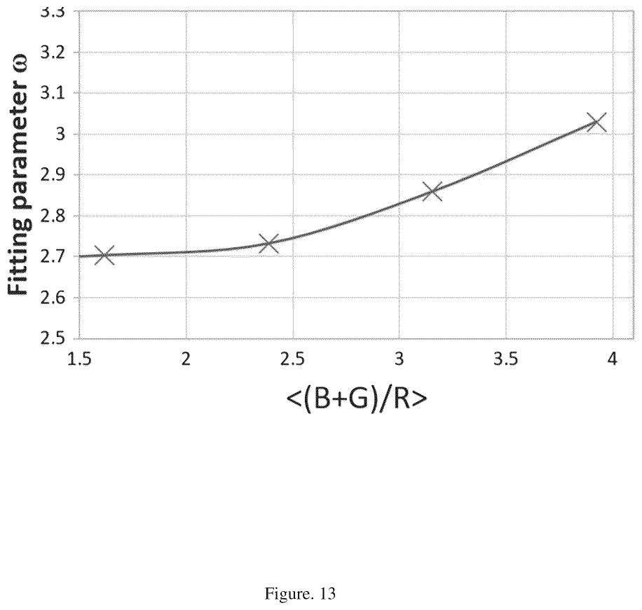

The diameter distribution (log-normal) average, w, exhibits a very clear trend as function of depth and as summarized in FIG. 13.

The premise behind the aforementioned results for the human endometrium is that they are not the only possible configuration for the BV network as function of depth, nor of the progression in time thereof. In order to corroborate these observations, we repeated the calculation for the four swine we had data for. By histological assessment, the swine were on their cycle days 2,8,14,16 (in a 21-day cycle). Seventy-three ex-vivo endometrial surface images of the four swine under 2× magnification were used for the following analysis. FIGS. 12A-12E show the BV diameter PDF of all the swine regardless of their cycle day. The upper most layer (the lower most panel) was somewhat contaminated by fresh surface blood.

In contrast to the human data, the “average” of the log distribution (w) becomes smaller as the layer deepens.

FIG. 13 shows this trend of the log-average (@) dependence on tissue depth with the positive slope as the layer becomes more superficial. Deeper layers (smaller (B+G)/R values have a smaller average width with respect to more superficial layers.

This observation proves the robustness of the method since the BV distribution of other mammals is known to be different to that of humans (see further in the discussion).

The statistical method presented here, is suggested to resolve some of the uncertainty in the endometrial angiogenesis process and establish clarity in the face of competing hypotheses.

It was demonstrated that by treating the BV population as a statistical ensemble, one can differentiate between different depths of BVs, using their color attribute as obtained in visual light images. This approach differentiates between evolution paths when BV diameter distribution is taken into account. The use of visible-light, non-destructive imaging also allows monitoring of the BV population evolution over a period of time within the very same tissue. The identification of a BV depth with its generation time (“age”), may or may not be justified according to the angiogenesis processes that have led to its creation.

In order to maximize the differentiating power of the method, the PDFs of the BVs (all together or within tissue layers) was split into a multiplication of their normalization factor, namely their total density, and their diameter distribution. These functions can then be determined with various mathematical models (Chappell et al., 2011) (Logsdon et al., 2014) that attempt to describe angiogenesis. For instance, their shape teaches whether new endometrial BVs are generated through sprouting, intussusception (a.k.a. splitting angiogenesis) or elongation. It was shown elsewhere (Or et al., 2022) that two-dimensional BVD alone (without splitting into depth layers) does not sufficiently characterize the endometrial evolution track.

There is a close relationship between the extracted PDFs and the underlying processes leading to their construction. For instance, a new endometrial layer has more sprouting or splitting potential locations than an older (deeper) endometrial layer and one may therefore expect a diameter distribution for the new endometrial layer to have a bigger normalization factor (“more vessels”) in addition to its shape to be heavily inclined towards smaller diameter values.

Alternatively, one may argue that older endometrial layer by virtue of its age, had a longer time to generate its BVs and therefore should be denser, with a higher normalization factor, but in keeping with the previous scenario-more inclined toward bigger diameter values.

An alternative scenario may argue that the endometrial tissue growth rate is much faster than the BV diameter growth rate and therefore the diameter distribution should be similar and narrow (with small diameters) throughout the depth of the endometrium.

A good example of the different statistical expression of processes can be demonstrated if we compare (1) elongation (Gambino et al., 2002) and (2) intussusception, where each one represents a single mechanism of angiogenesis.

In the case of elongation, the BV diameter distribution should stay relatively constant, and if elongation occurs at all endometrial layers, deeper layers should have a different normalization factor (namely BVD) yet a consistent normalized distribution (“shape”). On the contrary, if intussusception rules, a deeper layer that has already undergone splitting and the diameter distribution would lean towards smaller diameter values in comparison to the newly generated upper layer with thicker BVs.

In reality, probably all four possible processes described in the introduction contribute to the BV plexus but there may well be different weightings for different processes throughout the various phases of the menstrual cycle. The method we presented here may be used to identify and track intussusceptive angiogenesis, which has so far been beyond reach in human studies and mostly observed in animal data (Du Cheyne et al., 2021). The current statistical, time-evolution approach can now be combined with other methods to quantify its contribution to the overall angiogenesis process. The differential weightings may be the reason for the contrasting behavior of the swine BV PDF as a function of depth versus the human data as shown in this paper. This differential PDF behavior adds to the body of knowledge regarding differences between different mammalian species (mice, rhesus macaques, ewes) when it comes to angiogenesis (see e.g., (Chappell et al., 2012; Girling et al., 2007) and references therein).

Examination of FIG. 8, FIG. 9 and FIG. 12 revealed a clear trend that shouldn't necessarily exist a-priori. It is only due to the correct identification of the “color” for the BV with the “depth” coordinate that lends meaning to it.

In the swine case of FIG. 12 deeper layers (top panels) show a tail of relatively large diameter vessels. Moving to more superficial layers (downward on the plot) this “tail” diminishes and thus, both the average size diameter goes to smaller values (plot maximum goes to the right) and the standard deviation becomes smaller (plots become narrower).

The human sample exhibits the same trend of a shortening distribution “tail” as BVs closer to the tissue surface are considered. However, unlike the swine samples, the tail continues to much higher diameters. In view of the fact that human cycle is longer (28 days) than the swine's (21 days) this makes logical sense. It may reflect, for instance, the difference between the human menstrual cycle and an animal estrous one.

From the measurement perspective, even if the tissue effective absorption coefficients are different for swine and human, or the utilized light sources are different, the method is still self-referenced. The differences in absorption and illumination will globally affect the attenuation for each “color/depth” layer but will not affect the trend.

The immediate practical use, therefore, of the proposed method could be identification of abnormal evolution of the BV network that leads to various pathologies. Thus, as demonstrated here, even endometrial dating may be achieved through the identification of the relative BV population at different endometrial tissue depths. In combination with other digital, in-vivo, imaging and calculations, such endometrial dating may be more accurate than the traditional histological methods (Acosta et al., 2000; Dubowy et al., 2003; Murray et al., 2004; Noyes et al., 1950).

1. A method for characterizing at least one feature of blood vessels at different depths within a tissue of a mammalian subject, the method comprising:
(i) identifying blood vessels in an image obtained from a surface of said tissue,
(ii) determining for the identified blood vessels one or more of at least one feature of said identified blood vessels, and a color index ( [symbol] ), said color index differs in different depths within said tissue.

2. The method of claim 1, wherein said color index is different at the tissue surface and at a different depth within said tissue.

3. The method of claim 1, wherein said depths within said tissue is at most about 10 cm measured from said tissue surface.

4. The method of claim 1, wherein said identifying blood vessels comprises segmenting said blood vessels in said image.

5. The method of claim 1, wherein said determining at least one feature comprises (i) calculating a probability distribution function (PDF) for said at least one feature, and/or (ii) obtaining a statistical ensemble for said at least one feature in a population of blood vessels.

6. (canceled)

7. The method of claim 1, wherein said at least one feature comprises at least one of blood vessel diameter, blood vessel width (w), blood vessel length, blood vessel degree of tortuosity, oxygen saturation level, flow pattern or any combination thereof.

8. (canceled)

9. The method of claim 1, wherein said color index is calculated (i) from the blood vessels color band and/or (ii) from a combination of the blood vessels red color, green color and red color.

10. (canceled)

11. The method of claim 9, wherein said color index is calculated from a combination of R_{avg}, G_{avg} and B_{avg}, optionally wherein said color index is (i) calculated as (B_{avg}+G_{avg})/R_{avg} (Formula I) and/or (ii) calculated as B_{avg}/R_{avg} (Formula II).

12.-13. (canceled)

14. The method of claim 1, comprising obtaining an image of said tissue, optionally wherein said image is obtained by visible light imaging and/or infra-red-light imaging.

15. (canceled)

16. The method of claim 1, for determining (i) spatial coordinates of the blood vessels within the tissue, (ii) spatial correlation of the at least one feature of the blood vessels and/or of the spatial coordinates of the blood vessels, and/or (iii) directional correlation of at least one feature of the blood vessels and/or of the spatial coordinates of the blood vessels

17.-18. (canceled)

19. The method of claim 1, for characterizing a physiological process in said tissue, optionally wherein said physiological process is (i) differentiation, angiogenesis or apoptosis and/or (ii) associated with changes in blood vessels over time.

20.-21. (canceled)

22. The method of claim 1, comprising determining said at least one feature in at least two temporarily separated time points to monitor changes in blood vessels in said different depths within said tissue over time.

23. The method of claim 1, wherein said tissue is associated with changes in blood vessels with time.

24. The method of claim 23, wherein said changes in blood vessels comprise enhanced growth or destruction of blood vessels in said tissue.

25. The method of claim 1, wherein said tissue comprises at least a portion of said blood vessels that can be viewed from outside the tissue.

26. The method of claim 1, wherein said tissue is selected from the group consisting of: liver, kidneys, lungs, brain, heart, intestine, muscles, skin, a retina, uterine and a cancerous tissue, optionally wherein said tissue is uterine.

27. (canceled)

28. The method of claim 1, for (i) monitoring menstrual cycle in said subject, (ii) diagnosis infertility or infertility-related conditions in said subject, (iii) selecting the timing of embryo transfer and implantation, (iv) evaluating receptivity of the endometrium to embryo implantation in a subject, (v) determining suitability and timing for embryo transfer and implantation in a uterine of a subject, and/or (vi) characterizing said blood vessel population at different times of the menstrual cycle.

29.-33. (canceled)

34. The method of claim 1, being (i) an ex-vivo method and/or (ii) an in vivo method.

35. (canceled)

36. The method of claim 1, wherein said mammalian subject is (i) considered to undergo or in the process of in vitro fertilization (IVF) and/or (ii) diagnosed with a condition associated with destruction of blood vessels in said tissue.

37. (canceled)

38. The method of claim 1, for determining a pathological condition of a subject, optionally wherein said subject (i) is diagnosed with a condition associated with enhanced growth of blood vessels in said tissue and/or (ii) is suffering from a proliferative disorder.

39.-40. (canceled)
